(19) United States
(12) Patent Application Publication
(10) Pub. No.: US 2002/0151714 A1
(43) Pub. Date: Oct. 17, 2002

(54) PROCESS FOR PREPARING 1,4-DISUBSTITUTED PIPERIDINE COMPOUNDS

(76) Inventors: MAX REY, ZURICH (CH); STEFAN GLADOW, BUCHS (CH)

Correspondence Address: LORUSSO & LOUD, 440 COMMERCIAL STREET, BOSTON, MA, 02109

(21) Appl. No.: 09/380,835

(22) PCT Filed: Mar. 6, 1998
(86) PCT No.: PCT/CH98/00091

(30) Foreign Application Priority Data
Mar. 11, 1997 (CH) 571/97

(51) Int. Cl. C07D 211/68; C07D 211/80; C07D 213/02

(52) U.S. Cl. 546/93; 546/194; 546/203

(57) ABSTRACT

The invention concerns a process for preparing 1,4-disubstituted piperidine compounds of formula (I), in which Y =-(CH2) n , n being 0. 1. 2 or 3; oxygen; sulphur; vinyl, -CH 2 -0-; -0-CH 2; -CH 2 -S- or -S-CH 2 , and R and Z mean substituents known per se. Compounds in which Z means -CH 2 -CH 2 - are produced in that the correspondingly substituted 5,6-dihydro- 11 H -benzo[5,6]-cyclohepta-[1,2-b] pyridin-11-one compound is reacted with the corresponding 1-substituted 4-piperidone in a single process step by means of reductive dimerization in the presence of a finely dispersed metal of the IVth and/or Vth and/or VIth subgroup of the periodic table of elements or a low-valent oxidation stage of a corresponding metal compound of this type, the finely dispersed metal or the low-valent oxidation stage being produced in situ by means of a reducing agent and in the presence of an inert solvent. Preferably 4-(5.6-dihydro- 11 H -benzo-[5,6] cyclohepta[1 ,2-b] pyridin-11-ylidene)-1-piperidine compounds are produced.

Process for preparing 1, 4-disubstituted piperidine compounds

The present invention concerns a new process for producing 1,4-disubstituted piperidine compounds, in particular 4-(5-6-dihydro-11 H-benzo-[5,6]-cyclohepta-[1 ,2-b] pyridine- 11-ylidene)-1-piperidine compounds. The compound 4-(8-chloro-5,6-dihydro-11 H-benzo-[5,6]-cyclohepta-[1 ,2-b] pyridine-11-ylidene)-1-piperidine carboxylic acid ethyl ester (loratidine) has acquired particular significance as an H₁ antihistamine. Different processes for producing these compounds are described in the literature. However, the known processes have diverse disadvantages.

U. S. Pat. No. 4,731,447 describes a multiple-stage process, which, among other things, includes a Grignard reaction, in which it is necessary to work with an intermediary protective group on the piperidine nitrogen. The total yield is reduced because of the number of process steps which are, in part, difficult to perform. The use of n-butyl lithium described there requires extreme caution, as well as cyclization in super-acid conditions at very low temperatures. The process requires expensive reagents and also creates environmental problems. The other known processes also have similar disadvantages.

The process in accordance with the present invention on the whole has only a few process steps and requires no intermediate protective group on the piperidine nitrogen, a comparatively high yield being achieved. The process in accordance with the invention has no critical process steps for production. Also no reagents or solvents to be characterized as toxic, but at most slightly toxic or irritating, are used, so that all reactions could be carried out at ordinary reaction temperatures and in ordinary facilities. All products obtained in the process occur in crystalline form. No environmentally hazardous substances are used, created, or formed as intermediate products. The metals used, titanium and zinc, precipitate out of the reaction as non-toxic and easily reusable or disposable titanium oxides and zinc(II)tetramine complexes.

The present invention is defined in the Patent Claims. In particular, the present invention concerns a process for creating 1,4-disubstituted piperidine compounds of formula (1) in which R independently of one another mean hydrogen, fluorine, chlorine, bromine, straight-chain or branched (C₁- C₅) - alkyl, which in a given case is substituted with fluorine, chlorine, or bromine, with a (C₁- C₅) - alkyl-ether group and/or with phenyl; straight-chain or branched (C₂ - C5) - alkenyl, which in a given case is substituted with fluorine, chlorine, or bromine, with a (C₁- C₅) - alkyl ether group and/or phenyl; phenyl, which in a given case is substituted with fluorine, chlorine, bromine, (C₁- C₅) - alkyl, -COOH, (C₁- C₅) -alkyl ester, -NH2, a mono- (C₁- C₅) - alkyl substituted amine and/or a di-(C₁- C₅) - alkyl substituted amine; a hetero-aromatic, which is bonded directly or via straight-chain or branched (C₁- C₅) - alkylene to the pyridine and/or the phenyl ring, and contains a nitrogen atom and/or a sulfur atom and/or 1, 2, or 3 nitrogen atoms and contains a nitrogen atom and/or a sulfur atom and/or 1, 2, or 3 nitrogen atoms and a 5- or 6-member ring system which in a given case is substituted with fluorine, chlorine, bromine, (C₁-C₅) - alkyl, -COOH, (C₁- C₅) - alkyl ester, -NH2, a mono- (C₁- C₅) - alkyl substituted amine and/or a di-(C₁- C₅) - alkyl substituted amine, or two R substituents bonded to the same ring form an aromatic or hetero-aromatic ring, which in a given case is substituted with fluorine, chlorine, bromine, (C₁- C₅) - alkyl, -COOH, (C₁- C₅) - alkyl ester, -NH2, a mono- (C₁- C₅) - alkyl substituted amine and/or a di-(C₁- C₅) - alkyl substituted amine;

Y means -(CH₂)ₙ-, in which n =0, 1, 2, or 3; oxygen, sulfur; vinyl; CH2-0; -0-CH2; -CH2-or -S-CH₂;

Z independently of one another mean hydrogen, -C(O)R′; -C(O)OR′; -O)S(O)R²; or one of the meanings of R′; R¹ independently of one another mean straight-chain or branched (C₁- C₅) - alkyl, which in a given case is substituted with fluorine, chlorine, or bromine, with a (C₁- C₅) - alkyl ether group, and/or with phenyl; straight-chain or branched (C₂ - C₅) - alkenyl, which in a given case is substituted with fluorine, chlorine, or bromine, with a (C₁- C₅) - alkyl ether group, and/or phenyl; ; phenyl, which in a given case is substituted with fluorine, chlorine, bromine, (C₁- C5) - alkyl, -COOH, (C₁- C5) - alkyl ester, -NH₂, a mono- (C₁-C5) - alkyl substituted amine and/or a di-(C₁- C5) - alkyl substituted amine; a hetero-aromatic, which is bonded directly or via straight-chain or branched (C₁- C₅) - alkylene to the pyridine and/or the phenyl ring, and contains a nitrogen atom and/or a sulfur atom and/or 1, 2, or 3 nitrogen atoms and contains a nitrogen atom and/or a sulfur atom and/or 1, 2, or 3 nitrogen atoms and a 5- or 6-member ring system which in a given case is substituted with fluorine, chlorine, bromine, (C₁- C₅) - alkyl, -COOH, (C₁- C₅) - alkyl ester, -NH₂, a mono- (C₁- C₅) - alkyl substituted amine and/or a di-(C₁ - C₅) - alkyl substituted amine, or straight-chain or branched (C₁- C₅) - alkyl, which is substituted by such a hetero-aromatic.

R² means one of the meanings of R¹, or a bridged saturated isocyclic system, which preferably is derived from camphor sulfonic acid; which is characterized by the fact that a compound of formula (II) (II) in which the substituents R and Y have the meanings cited above, which a compound of formula (III) (III) in which Z has the meaning specified above, is brought to react in a single process step by means of reductive dimerization (i) in the presence of a finely dispersed metal compound of the IVth and/or Vth and/or VIth subgroup of the periodic table of elements or a low-valent oxidation stage of such a corresponding metal compound, (ii) the finely dispersed metal or the low-valent oxidation stage being produced in situ by means of a reducing agent and (iii) in the presence of an inert solvent, the reducing agent being chosen from the group of alkali metals, the metals of the IInd main group or lInd sub-group of the periodic table, alloys of these metals, alloys of these metals with zinc, zinc-copper aloys, inclusion compounds of such metals with carbon, preferably potassium-graphite inclusion compounds, metal hydrides, salts of naphthalidone ions or of higher polycyclic aromatics and the solvent being chosen from the group of inert ethers, preferably 1,4-dioxane, 1,2-dimethoxyethane, tetrahydrofurane, diethylene glycol dimethyl ether, tert.-butyl-methyl ether, or the group of nitrogen-containing unsaturate hetero-aromatics, preferably pyrindine or tertiary amines, preferably triethyl amine.

The smooth conversion of amide, sulfonamide, and urethane substituted piperidine derivatives even in the case of long reaction times is surprising for the reaction in accordance with the invention, in particular since it is known that these functional groups are decomposed slowly under the chosen reaction conditions (cf. John E. McMurry, Carbonyl-Coupling, Reactions Using Low-Valent Titanium, Chem. rev. 89, p. 1513-1524 (1989), in particular pages 1515 ff). However, for the reactions described in this patent no decomposition can be observed even in the case of very long reaction times (>96 hours). In general, only a few successful coupling reactions ofcarbonyl compounds, which are substituted with the functional groups designated above, with low-valent titanium compounds, are known in the literature. Equally surprising are the high yields in the case of using titanium tetrachloride (TiCl₄). In the literature preference is given to the very unstable, oxidation-sensitive titanium trichloride (TiCl₃), which is little suited for industrial production. Yields which are comparably as good as those described in this patent are found in the literature in particular with the use of titanium trichloride.

Titanium, zirconium, vanadium, molybdenum., tungsten, and uranium are particularly well suited as metals, respectively metal compounds of the IVth, Vth, and VIth sub-groups of the periodic table, preferably halogen compounds thereof, preferably the chloride being used. The use of titaniun tetrachloride is preferred, a low-valent stage ofthis compound, respectively of these named compounds, being created in situ by means of a reducing agent. Preferably zinc or alkali metals, preferably zinc, lithium, sodium, or potassium; metals of main group II or sub-group II of the periodic table, in particular magnesium or calcium; alloys containing zinc, lithium, sodium, potassium, magnesium, and/or calcium; zinc-copper alloys; inclusion compounds of zinc, lithium, sodium, potassium, magnesium, and/or calcium with carbon, in particular alkali metal hydrides or lithium aluminum hydride; salts of naphthalide anions, preferably lithium naphthalide or sodium naphthalide, are used as reducing agents.

Preferably inert ethers, preferably 1,4-dioxane, 1,2-dimethoxyethane, diethylene glycol dimethyl ether (diglyme), tert.-butyl methyl ether, as well as nitrogen-containing unsaturated hetero-aromatics, preferably pyridine or tertiary amides, preferably triethyl amine, are used as solvents.

The ratio of the compound of formula III) (in mol- equivalents) to the coupling metal compound (in equivalents, oxidation state +111 or +IV) preferably amounts to 3: 1 to 1: 100, preferably 1 3, in performing the reaction.

The ratio of the reducing agent (in reduction equivalents) to the coupling metal compound (in equivalents, oxidation stage +111 or +IV) preferably amounts to 1: 2 to 100: 1, preferably 2 1, in performing the reaction.

The reaction temperature amounts to 0° C. to 200° C., preferably 10° C. to 100° C., and in particular around 20° C. to 70° C.

In the cited formulas of compounds (I) to (II), R means, preferably and independently of one another, hydrogen, fluorine, chlorine, bromine, methyl, or trifluoromethyl, particularly preferably R means, independently of one another, hydrogen, fluorine, or chlorine. The compound of formula (I) preferably has two substituents, which are different from hydrogen, one substituent R being on the pyridine ring and one substituent R being on the benzene ring, and the latter substituent preferably being fixed in 8-position. Preferably the compound of formula (I) has only one substituent R, which is found preferably in 8-position, and preferably means fluorine or chlorine.

The preferred meaning of Y is -CH₂ -CH₂; Preferably R² means (C₁- C₅) - alkyl and, in particular, ethyl. Preferably R² means (C, - C₅) - alkyl, benzyl, vinyl, or dimethyl amino (-N(CH₃)₂), in particular methyl. Preferably Z means -C(O)R¹; -C(O)OR¹, preferably -C(O)OR¹. For the most part preferably Z means a radical of the formula -C(O))-C₂H₅. (C₁- C₅) - alkyl means methyl, ethyl, propyl, isopropyl, n-butyl, isobutyl, tert.-butyl, n-pentyl, or branched pentyl, preferably methyl, ethyl, or propyl, preferably methyl or ethyl. (C₂ - C₅) -alkenlyl preferably means (C₂ - C₃) - alkenyl and preferably (C₃) - alkeiiyl. 8-chlorine-substituted compounds and, in particular, the compound 4-(8-chloro-5,6-dihydro-11 H-benzo-[5,6]-cyclohepta-[1,2-b]pyridine-11-ylidene)-1 -piperidine carboxylic acid ethyl ester, are preferred compounds of formula (1)..

The production of the compound can take place in four stages in formula (I), in which Y is -CH₂ -CH₂. In a first stage a cyanide-substituted compound of formula (IV) (IV)

in which the substituents R have the meaning specified above, is brought to react with a benzaldehyde compound of formula (V) (V)

in which R has the meaning cited above, by means of a base-induced aldol condensation, preferably in the presence of potassium tert.-butylene, with simultaneous saponification of the nitrile group to the amide, to the compound of formula (VI): (VI)

In the second stage the double bond of the compound of formula (VI) is hydrated on a palladium catalyst, without the other substituents, which are found on the benzoring, being hydrated or split off. In this way the amide of the compound of formula (VII) is obtained: (VII)

In the third stage the amide of formula (VII) is hydrolyzed in a basic medium, the carboxylic acid of the compound of formula (VIII) being obtained. (VIII)

In the fourth stage the compound of formula (VIII) is converted into the corresponding acid chloride by means of thionyl chloride (SOCl₂), and cyclized to the tricycle of formula (IX) by means of a Friedel-Krafts reaction in the presence of aluminum chloride. (IX)

In the compound of formula (IV) R=hydrogen and 3-chloro-benzaldehyde is used as a compound of formula (V), thus 8-chloro-5,6-dihydro-11H-benzo-[5,6]-cyclohepta-L [1,2-b]b]pyridine-11-one is obtained. If this compound is made to react with 1-(ethoxycarbonyl)-4-piperidone, that is a compound of formula (III), in which Z means ethoxycarbonyl, 4-(8-chloro-5 ,6-dihydro-11H-benzo-[5 ,6]-cyclohepta-[ I,2-b]pynidine- 11-ylidene)-1-piperidine carboxylic acid ethyl ester (loratadine) is obtained. The production of this compound is a preferred embodiment of the present invention.

Compounds of formula (II), in which Y is oxygen, sulfur, vinyl, CH₂-O; -O-CH₂; -CH₂-, or -S-CH₂, can be produced in a way known per se. The following examples explain the invention.

### Example I 4-(5,6-dihydro-11-benzo-[5,6-cyclcohenta-] 2pyridine -11 -ylidene)-1-piperidine carboylic acid ethyl ester

10.11 g (155 mmol) of powdered zinc and 13.04 g, (69 mmol) of titanium tetrachloride are heated to boiling in 65 g of absolute tetrahiydrofuirane (THF) under a protective gas atmosphere. As soon as the solution is colored black, it is cooled to room temperature. 5.25 g(25 mmol) of 5,6-dilhydro- 11H-benzo-[5,6]-cyclohepta-[ I,2-b]pyridine- 11-one and 4.25g (25 mmol) of 1-(ethioxycarboniyl)-4-piperdine are to be added. The preparation is stirred over night at room temperature and then 5 hours at 40° C. The preparation is concentrated in 60 g of ethyl acetate, dissolved in 60 g of ethyl acetate, and diluted with 100 g of a saturated aqueous solution of ethylene diamino-tetra acetic acid-tetra sodium salt-dihydrate. After the heat development dies down, the organic phase is separated, and the aqueous phase is rewashed two times with 20 g of ethyl acetate. The aqueous phase then is treated with 30% hydrogen peroxide solution, until the gray-colored low-valent titanium compounds have reacted completely to the white titanium (IV) dioxide, and discarded. The combined organic phases are dried over sodium sulfate, filtered, and concentrated to dry. In this case the product is separated as a semicrystalline syrup. The latter is dissolved in 40g of ethyl acetate / diiusopropyl ether and heated to the reflux. After the addition of activated charcoal and hot filtration, the product precipitates out in the form of colorless crystals (in a given case after seeding). Yield: 5,9% (68%); HPLC-purity 94%, melting point 108° C.-109° C.

### Example2 4-(8-chloro-5,6-dihydro- 11H-ben70-[5,6]-cyclohepta-[I,.2-b]pyridine- 11-yli dene)-1 -piperidine carboxylic acid ethyl ester

In accordance with Example 1, 40.0 g (160 mmol) of 8-chloro-5,6-dihydro-11 H-benzo-[5,6]-cyclohepta-[1,2-b]pyridine-11-one and 27.4 g (160 mmol) 1-(ethoxy)-4-piperidone are brought to react with a coupling reagent consisting of 53.6 g (820 mmol) zinc and 75.9g (400 mmol) of titanium tetrachloride. After cooling and crystallization out of ethyl acetate / diisopropyl ether (in a given case after seeding), 4-(8-chloro-5,6-dihydro-1 lH-benzo-[5,6]-cyclohepta-[1,2-b]pyridine- 11-ylidene- 1 -piperidine carboxylic acid ethyl ester is obtained in a yield of 47.2 g (75%); HPLC purity 97%; as colorless crystals, melting point 136° C - 138° C.

### Example3 4-(8-fluoro-5 ,6-dihydro- 11 H-benzo -[5,6]-cyclohepta-[ 2-b]pyri dine- 11 -yl idene)- 1 -piperidine carboxylic acid ethyl ester

In accordance with Example 1, 27.35 g (119 mmol) of 8-flioro-5,6-dihydro- IH-benzo-[5,6]-cyclohepta-[1,2-b]pyridine- 11-one and 20.0 g (117 mmol) 1-(ethoxy)-4-piperidone are brought to react with a coupling reagent consisting of 37.0 g (566 mmol) zinc and 50.9g (268 mmol) of titanium tetrachloride. After cooling and crystallization out of ethyl acetate / diisopropyl ether (in a given case after seeding), 4-(8-fluoro-5,6-dihydro-1 IH-benzo-[5,6]-cyclohepta-[1,2-b]pyridine-11-ylidene-1-piperidine carboxylic acid ethyl ester is obtained in a yield of 38.0 g (67%); HPLC purity 96%; as colorless crystals, melting point 119° C. - 121° C.

### Example 4 4-(8-chloro-5 6-dihydro- 11H-benzo-[5,6]-cycloheptant 2-b]pyridine- 11-vlidcne)-11-acetyl piperidine

31.7g of zinc-copper alloy and 24.1g (156 mmol) of titanium chloride are heated to boiling in 175 g of absolute tetrahydrofuranie (THF) under protective gas atmosphere. As soon as the solution is colored black, it is cooled to room temperature. 15.31g (63 mmol) 8-chloro-5,6-dihydro-11 H -benzo-[5,6]-cyclohcpta-[1,2-b]pyridine-11-one and 8.90 g (63 mmol) of 1-acetyl-l- piperidonc are to be added. It is stirred for 3 hours at room temperature and then for 6 hours at 50° C. The preparation is concentrated, dissolved in 120 g of ethyl acetate, and diluted with 250 g of a saturated aqueous solution of ethylene diamino-tetra acetic acid-tetra sodium salt-dihydrate. After the heat development dies down, the organic phase is separated and the aqueous phase is rewashed two times with 30 g of ethyl acetate. The aqueous phase then is treated with 30% hydrogen peroxide solution, until the gray-colored low-valent titanium compounds have reacted corhpletely to the white titanium (IV) dioxide, and discarded. The combined organic phases are dried over sodium sulfate, filtered, and concentrated to dry. After cooling and crystallization out of ethyl acetate / diisopropyl ether (in a given case after seeding), 4-(8-chloro-5 ,6-dihydro- 11 H-benzo-[5,6]-cyclohepta-[ 1 ,2-b]pyridine- 11 -ylidene-1)-1-acetyl-piperidine is obtained in a yield of 16.00 g (72%); HPLC purity 96%; as colorless crystals, melting point 161° C. - 162° C. Remark: all melting points in examples 1 to 4 are uncorrected.

1. A process for creating 1,4-disubstituted piperidine compounds of formula (1) (I) in which
R independently of one another mean hydrogen, fluorine, chlorine, bromine, straight-chain or branched (C₁- C₅) - alkyl, which in a given case is substituted with fluorine, chlorine, or bromine, with a (C₁- C₅) - alkyl-ether group and/or with phenyl; straight-chain or branched (C₂ - C₅) - alkenyl, which in a given case is substituted with fluorine, chlorine, or bromine, with a (C₁- C₅) - alkyl ether group and/or phenyl; phenyl, which in a given case is substituted with fluorine, chlorine, bromine, (C₁- C₅) - alkyl, -COOH, (C₁- C₅) alkyl ester, -NH2, a mono- (C₁- C₅) - alkyl substituted amine and/or a di-(C₁- C₅) - alkyl substituted amine; a hetero-aromatic, which is bonded directly or via straight-chain or branched (C₁- C₅) - alkylene to the pyridine and/or the phenyl ring, and contains a nitrogen atom and/or a sulfur atom and/or 1, 2, or 3 nitrogen atoms and contains a nitrogen atom and/or a sulfur atom and/or 1, 2, or 3 nitrogen atoms and a 5- or 6-member ring system which in a given case is substituted with fluorine, chlorine, bromine, (C₁-C₅) - alkyl, -COOH, (C₁- C₅) - alkyl ester, -NH2, a mono- (C₁- C₅) - alkyl substituted amine and/or a di-(C₁- C₅) - alkyl substituted amine, or two R substituents bonded to the same ring form an aromatic or hetero-aromatic ring, which in a given case is substituted with fluorine, chlorine, bromine, (C₁- C₅) - alkyl, -COOH, (Cl - C₅) - alkyl ester, -NH2, a mono- (C₁- C5) - alkyl substituted amine and/or a di-(C₁- C₅) - alkyl substituted amine;
Y means -(CH₂), in which n =0, 1, 2, or 3; oxygen, sulfur; vinyl; CH₂-O; -O-CH₂; -CH₂-, or -S-CH₂;
z independently of one another mean hydrogen, -C(O)R¹; -C(O)OR¹; -O)S(O)R²; or one of the meanings of R¹;
R¹ independently of one another mean straight-chain or branched (C₁- C₅) - alkyl, which in a given case is substituted with fluorine, chlorine, or bromine, with a (C₁- C₅) - alkyl ether group, and/or with phenyl; straight-chain or branched (C₂ - C₅) - alkenyl, which in a given case is substituted with fluorine, chlorine, or bromine, with a (C₁- C5) - alkyl ether group, and/or phenyl; phenyl, which in a given case is substituted with fluorine, chlorine, bromine, (C₁- C₅) - alkyl, -COOH, (C₁- C₅) - alkyl ester, -NH₂, a mono- (C₁-C₅) - alkyl substituted amine and/or a di-(C₁- C₅) - alkyl substituted amine; a hetero-aromatic, which is bonded directly or via straight-chain or branched (C₁- C₅) - alkylene to the pyridcine and/or the phellyl ring, and contains a nitrogen atom and/or a sulfur atom and/or 1, 2, or 3 nitrogen atoms and contains a nitrogen atom and/or a sulfur atom and/or 1, 2, or 3 nitrogen atoms and a 5- or 6-member ring system which in a given case is substituted with fluorine, chlorine, bromine, (C₁- C₅) - alkyl, -COOH, (C₁- C₅) - alkyl ester, -NH₂, a mono- (C₁- C₅) - alkyl substituted amine and/or a di-(C₁- C₅) - alkyl substituted amine, or straight-chain or branched (C₁- C₅) - alkyl, which is substituted by such a hetero-aromatic.
R² means one of meanings of R¹, or a bridged saturated isocyclic system, which preferably is derived from camphor sulfonic acid;
wherein a compound of formula (II) (II)
in which the substituents R and Y have the meanings cited above, with a compound of formula in which Z has the meaning specified above, is brought to react in a single process step by means of reductive dimerization (i) in the presence of a finely dispersed metal compound of the JVth and/or Vth and/or VIth subgroup of the periodic table of elements or a low-valent oxidation stage of such a corresponding metal compound, (ii) the finely dispersed metal or the low-valent oxidation stage being produced in situ by means of a reducing agent, and (iii) in the presence of an inert solvent, the reducing agent being chosen from the group of alkali metals, metals of the IInd main group or IInd subgroup of the periodic table, alloys of these metals, inclusion compounds of such metals, or of higher polycyclic aromatics, and the solvent is chosen for the group of the inert ethers or the group of nitrogen-containing, unsaturated hetero-aromatics or the tertiary amines.

2. The process in accordance with Patent claim 1, wherein R independently of one another means hydrogen, fluorine, chlorine, bromine, methyl, or trifluloromethyl.

3. The process in accordance with Patent claim 1, wherein R independently of one another means hydrogen, fluorine, or chlorine.

4. The process in accordance with one of the Patent claims 1 or 2, wherein the compound of formula (1) has two substituents R, which are different from hydrogen, one substituent R being on the pyridine ring and one substituent R on the benzoring.

5. The process in accordance with Patent claim 4, wherein the substituent R is fixed on the benzoring in 8-position.

6. The process in accordance with one of the Patent claims 1 to 3, wherein the compound of formula (1) only has a single substituent R, which is different from hydrogen, this substituent R being fixed in R-position.

7. The process in accordance with one of the Patent claims 1 to 6, wherein
Y means -CH₂ -CH₂;
R¹means (C₁- C₅)-alkyl , preferably ethyl;
R² means (C₁- C₅) - alkyl, benzyl, vinyl, or dimethyl amino, preferably methyl;
Z means -C(O)R¹;-C(O)OR¹, preferably -C(O)OR¹, and preferably -C(O))-C₂H₅.

8. The process in accordance with one of the Patent claims 1 to 7, wherein a halogen compound is used as the metal compound.

9. The process in accordance with Patent claim 8, wherein a chloride of titanium, zirconium, vanadium, molybdenum, tungsten, and/or uranium is used as metal compound.

10. The process in accordance with Patent claim 8, wherein titanium chloride is used as a metal compound and a low-valent stage of this compound is created in situ by means of a reducing agent.

11. The process in accordance with one of the Patent claims 1 to 9, wherein zinc, lithium, sodium, potassium, magnesium, or calcium or alloys containing zinc, lithium, sodium, potassium, magnesium, and/or calcium, calcium hydride, sodium borhydride, or lithium aluminum hydride is used as reducing agent.

12. The process in accordance with one of the patent claims 1 to 9, wherein an alloy of an alkali metal, a metal of the IInd main group, or the IInd subgroup of the periodic table with zinc, a zinc-copper alloy, or a potassium-graphite inclusion compound is the reducing agent.

13. The process in accordance with one of the patent claims 1 to 12, wherein 1,4-dioxane, 1,2-dimethoxyethane, tetrahydrofrane, diethylene glycol dimethyl ether, tert.-butyl-methyl ether, pyridine, or triethyl amine is used as solvent.

14. The process in accordance with one of the Patent claims 1 to 13, wherein the compound 4-(8-fluoro-5 ,6-dihydro- 11H-benzo-[5 ,6]-cyclohepta-[ ,2-b]pyridine- 1 -ylidene)- 1 -piperidine carboxylic acid ethyl ester is produced.

15. The compounds produced in accordance with one ofthe Patent claims 1 to 14
